(19) United States
(12) Patent Application Publication
(10) Pub. No.: US 2023/0355489 A1
(43) Pub. Date: Nov. 9, 2023

(54) MINERAL SUNSCREEN COMPOSITION

(71) Applicant: Edgewell Personal Care Brands, LLC, Chesterfield, MO (US)

(72) Inventors: Teresa M Fleming, Mahwah, NJ (US); Kelly L Ruffenach, Cedar Grove, NJ (US); Huong Bach, New Milford, NJ (US)

(21) Appl. No.: 17/906,797

(22) PCT Filed: May 5, 2021
(86) PCT No.: PCT/US2021/030824

§ 371 Date: Sep. 20, 2022

Related U.S. Application Data
(60) Provisional application No. 63/020,846, filed on May 6, 2020.

(51) Int. Cl. A61K 8/29; A61K 8/34; A61K 8/37; A61K 8/27; A61Q 17/04; A61K 8/891; A61K 8/85

(52) U.S. Cl. CPC A61K 8/29; A61K 8/345; A61K 8/375; A61K 8/27; A61K 8/37; A61Q 17/04; A61K 8/891; A61K 8/85; A61K 2800/28; A61K 2800/30

(57) ABSTRACT

A sunscreen composition includes a metal oxide sunscreen active, an emollient including a benzoate ester and an organosilicon compound, an emulsifier, and one or more other ingredients such as water. The metal oxide may be present in an amount between about 3.0 wt. % to about 20.0 wt. %, the benzoate ester may be present in an amount between about 5.0 wt. % to about 30.0 wt. %, the organosilicon compound may be present in an amount of about 3.5 wt. %, the emulsifier may be present in an amount between about 2.0 wt. % to about 10.0 wt. %, and water may be present in an amount of 30.0 wt. % or more, based on the total weight of the sunscreen composition. The sunscreen composition may be in the form of lotion that does not foam and is rubbed in to contact the keratinous surface.

### CROSS-REFERENCE TO RELATED APPLICATIONS

This application claims priority to U.S. Provisional Patent Application No. 63/020,846, filed on May 6, 2020, the content of which is incorporated herein for reference in its entirety.

### TECHNICAL FIELD

Embodiments relate to sunscreen compositions, that may exhibit enhanced application and/or performance attributes, including a mineral sunscreen active and one or more emollient(s), humectant(s), emulsifier(s), film former(s), and/or other ingredient(s).

BACKGROUND

Ultraviolet (UV) solar radiation falling within the UVA region (320 nm to 400 nm) is damaging owing to its ability to reach deeper into the dermis layer as compared to UV solar radiation falling within the UVB region (290 nm to 320 nm). As such, UVA radiation may suppress immune function, lead to loss of skin elasticity, promote premature signs of aging, or lead to other undesirable health effects. Meanwhile, UVB radiation is absorbed by the epidermis layer to cause erythema and cellular mutations that damage the skin or underlying tissue. Indeed, the erythemal action spectrum reveals that 99% of erythema action resides in the UVB region and 1% resides in the UVA region.

Accordingly, sunscreen compositions include one or more sunscreen actives to protect keratinous substrates, such as skin and hair, from UV radiation. One traditional category of sunscreen actives is inorganic, or mineral, sunscreen actives. Sunscreen compositions that have a relatively high concentration of mineral sunscreen actives, however, may exhibit technical challenges related to its application or performance. For example, a concentration of mineral sunscreen actives >10.0 wt. % may induce drag in a sunscreen composition, may cause a sunscreen composition to be relatively harder to smooth into skin, and/or may cause a sunscreen composition to be relatively harder to spread onto skin. Thus, there is considerable room for improvement to provide a sunscreen composition exhibiting enhanced application and/or performance at relatively high amounts of mineral sunscreen actives.

### SUMMARY

Embodiments relate to sunscreen compositions including a sunscreen active, an emollient, a humectant, an emulsifier, a film former, and/or one or more other ingredients.

The sunscreen active may include a metal oxide selected from zinc oxide, titanium dioxide, or combinations thereof. The metal oxide may, for example, be present in an amount between about 3.0 wt. % to about 20.0 wt. %, based on the total weight of the composition.

In any of the preceding embodiments, the emollient may include a benzoate ester. In one example, the benzoate ester may include C₁₂-C₁₅ alkyl benzoate. In one example, the benzoate ester may be present in an amount between about 5.0 wt. % to about 30.0 wt. %, based on the total weight of the composition. For example, the benzoate ester may be present in an amount of 15 wt. % or more, based on the total weight of the composition.

In any of the preceding embodiments, the emollient may further include a diol, an oil, a fat, a lipid, a triglyceride, a siloxane, salicylic acid and/or derivatives thereof, or combinations thereof. In one example, the emollient may include plant-based oil, dimethicone, glyceryl stearate, butyloctyl salicylate, or combinations thereof. Thus, emollients may provide anti-foaming functionality and embodiments may include substantially non-foaming formats, completely non-foaming formats, lotion formats (e.g., sun milk, etc.), ingredients that dampen and/or minimize foaming, or combinations thereof.

The one or more other emollients may, for example, be present in an amount between about 0.1 wt. % to about 6.0 wt. %, based on the total weight of the composition. Moreover, the one or more other emollients may account for a reduction of a portion of an alkyl benzoate in the sunscreen composition. For example, caprylic/capric triglycerides may be present in an amount between about 5.0 wt. % to about 10.0 wt. % to account for a reduction of a portion of C12-C15 alkyl benzoate to a concentration of 5.0 wt. % to 10.0 wt. %.

In any of the preceding embodiments, the humectant may include a lipid, a polymer, or combinations thereof. In one example, the humectant may be selected from glycerin, octyldodecyl citrate crosspolymer, or combinations thereof. The humectant may, for example, be present in an amount between about 1.0 wt. % to about 20.0 wt. %, based on the total weight of the composition. For example, the humectant may be present in an amount between about 1.5 wt. % to about 7.5 wt. %, based on the total weight of the composition.

In any of the preceding embodiments, the emulsifier may include a nonionic emulsifier, a cationic emulsifier, an anionic emulsifier, an amphoteric emulsifier, or combinations thereof. In one example, the emulsifier may be selected from cetyl PEG/PPG-10/1 dimethicone, lauryl PEG-8 dimethicone, caprylic/capric triglyceride (and) stearalkonium bentonite (and) and propylene carbonate, or combinations thereof. The emulsifier may, for example, be present in an amount between about 2.0 wt. % to about 10.0 wt. %, based on the total weight of the composition.

In any of the preceding embodiments, the film former may include monomers, copolymers, cross polymers, and/or terpolymers of an organo-silicone hybrid, biopolymer, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. In one example, the film former may be selected from VP/eicosene copolymer, octyldodecyl citrate crosspolymer, or combinations thereof. The film former may, for example, be present in an amount between about 0.1 wt. % to about 5.0 wt. %, based on the total weight of the composition.

In any of the preceding embodiments, the one or more other ingredients may include a preservative system, a thickener, a solvent, an extract, a colorant, a light stabilizer, a dispersing agent, or combinations thereof. In one example, the one or more other ingredients may be present in an amount between about 0.01 to about 45.0 or more, based on the total weight of the composition. Water, for example, may be present in an amount of 30 wt. % or more, based on the total weight of the composition.

Embodiments further relate to a method to protect a keratinous surface from UV radiation including contacting the keratinous surface with the sunscreen composition of any of the preceding embodiments. Embodiments further relate to a method to manufacture a sunscreen composition of any the preceding embodiments, comprising mixing a sunscreen active with an emollient, a humectant, an emulsifier, a film former, and/or one or more other ingredients.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. Any numerical range recited herein is intended to include all sub-ranges subsumed therein, and such ranges are understood to include each and every number and/or fraction between the stated range lower and upper values. Moreover, the term “about” may refer to deviations of ±10%-20% unless otherwise stated.

Sunscreen Active(s)

A sunscreen composition includes a sunscreen active to substantially absorb, scatter, and/or deflect UV radiation on a keratinous substrate. A sunscreen active may include, for example, p-aminobenzoic acid (PABA) and derivatives thereof, avobenzone (also known as butylmethoxy dibenzoylmethane), 2,4-dihydroxybenzophe none, 2,2′,4,4′-tetrahydroxybenzophenone, oxybenzone, sulisobenzone, sulisobenzone sodium, 2,2′-dihydroxy-4,4′-dimethoxybenzophenone, 5-chloro-2-hydroxybenzophenone, dioxybenzone, sodium 2,2′-dihydroxy-4,4′-dimethoxybenzophenone-5,5′-disulfonate, 2-hydroxy-4-methoxy-4′-methyl-benzophenone, octabenzone, ethyl dihydroxypropyl-p-aminobenzoate, glyceryl-p-aminobenzoate, homosalate, methyl anthranilate, octocrylene, octyl dimethyl-p-aminobenzoate, isoamyl-p-methoxycinnamate, octyl methoxycinnamate, octyl salicylate, triethanolamine salicylate, 3-(4-methylbenzylidene) camphor, enzacamene, phenylbenzimidazole sulfonic acid, methylene bis-benzotriazolyl tetramethylbutyl phenol, 4-isopropyldibenzoylmethane, octisalate, bis-ethylhexyloxyphenol methoxyphenyl triazine, 4-isopropyl-dibenzoylmethane, metal oxides such as zinc oxide or titanium dioxide, terephthalylidene dicamphor sulfonic acid (also known as ecamsule), drometrizole trisiloxane, diethylhexylbutamido triazone, ethylhexyl triazone, cinoxate, ensulizole, bis-disulizole disodium, diethylamino hydroxybenzoyl hexylbenzoate, or combinations thereof.

Commercially available metal oxides may have various morphologies, including shape, surface topography, size, or combinations thereof. Metal oxides may be also be coated or uncoated. For example, metal oxide useful as sunscreen actives may have coatings composed of silicone polymers, fatty acids or salts thereof, oxides of silicon, aluminum, or zirconium, or combinations thereof. In one example, titanium dioxide particles may have an inner coating of aluminum hydroxide and an outer coating of isostearic acid. In addition, metal oxides may be dispersed in non-aqueous diluent such as, without limitation, isododecane, dodecane, tridecane, isohexadecane, pentadecane, or combinations thereof.

Preferably, a sunscreen composition provides protection against UVA and UVB radiation. For example, the sun protection factor (SPF) of a sunscreen composition may be greater than or equal to 15 and the UVA protection factor (UVAPF) may be 1/3 the SPF. Notably, a sunscreen active that provides protection against UVA and UVB radiation may include titanium dioxide, zinc oxide, or combinations thereof, which can be used in effective amounts to provide an SPF of 8 or more (in contrast to other uses including as colorants, etc.). In addition, a sunscreen composition may provide protection against damaging radiation within other regions in the electromagnetic spectrum, such as blue light (380 nm to 500 nm).

Thus, for example, a sunscreen composition may include one or more metal oxides (e.g., titanium dioxide, zinc oxide, combinations thereof, etc.). Additionally or alternatively, a sunscreen composition may include a sunscreen active selected only from metal oxides.

A sunscreen active may be present in an amount of about 1.0 wt. % to about 40.0 wt. %, based on the total weight of the sunscreen composition. A sunscreen active may, for example, be present in an amount of 0.45 wt. %, 1.0 wt. %, 5.0 wt. %, 6.0 wt. %, 11.0 wt. %, 15.0 wt. %, 20.0 wt. %, 30.0 wt. %, 44.0 wt. %, including every number and/or fraction therebetween, based on the total weight of the sunscreen composition. In one example, a sunscreen composition may include only a metal oxide sunscreen active such as, without limitation, only zinc oxide, titanium dioxide, zinc oxide and titanium oxide, etc. A sunscreen composition may, for example, include a metal oxide present in an amount between about 3.0 wt. % to about 20.0 wt. %, based on the total weight of the sunscreen composition.

Emollient(s)

A sunscreen composition may include an emollient to smooth and/or soften a keratinous substrate. Preferably, the emollient includes esters of benzoic acid and a corresponding alcohol. For example, a benzoate ester may include the ester of benzoic acid and an alkyl alcohol (C₁₂-C₁₅ alcohols). The emollient may, for example, be selected from a benzoate ester having relatively longer chain alkyl acetates such as, without limitation, lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, or combinations thereof. In one example, the molecular weight of the benzoate ester may be between, for example, about 234 to about 431 g/mol.

Additionally or alternatively, the emollient may include a diol. For example, the diol may include an aliphatic diol such as, without limitation, butylene glycol, hexylene glycol, 2,3-butanediol, or combinations thereof. In one example, the diol may include a 1,2-alkane diol such as, without limitation, propylene glycol, 1,2-butanediol, pentylene glycol, ethyl hexanediol, caprylyl glycol, decylene glycol, or combinations thereof.

Additionally or alternatively, the emollient may include an oil, a fat, a lipid, a triglyceride, or combinations thereof. For example, the emollient may include plant-based oil (e.g., nut oil, seed oil, fruit oil, etc.), mineral oil, fatty acids (e.g., animal oils, etc.), triglycerides, or combinations thereof. In one example, the emollient may include coconut oil, candlenut oil, caprylic/capric triglyceride, glyceryl stearate, or combinations thereof.

Additionally or alternatively, the emollient may include a compound having at least one siloxane (Si—O—Si linkage). For example, the emollient may include an organosilicone compound such as, without limitation, dimethicone. Additionally or alternatively, the emollient may include salicylic acid and/or derivatives thereof. For example, the emollient may include butyloctyl salicylate, C₁₂-C₁₅ alkyl salicylate, capryloyl salicylic acid, hexydodecyl salicylate, isocetyl salicylate, isodecyl salicylate, tridecyl salicylate, or combinations thereof.

An emollient may be present in an amount of about 0.1 wt. % to about 30.0 wt. %, based on the total weight of the sunscreen composition. An emollient may, for example, be present in an amount of 0.09 wt. %, 0.1 wt. %, 1.0 wt. %, 3.0 wt. %, 5.0 wt. %, 8.0 wt. %, 17.0 wt. %, 20.0 wt. %, 25.0 wt. %, 30.0 wt. %, 33.0 wt. %, including every number and/or fraction therebetween, based on the total weight of the sunscreen composition.

For example, a sunscreen composition may include C₁₂-C₁₅ alkyl benzoate in an amount greater than about 5.0 wt. % to less than 30.0 wt. %, based on the total weight of the sunscreen composition. One or more emollients may also be added to the sunscreen composition based on the amount of alkyl benzoate used in the sunscreen composition. For example, one or more emollients may be added in an amount sufficient to total about 30.0 wt. % of emollient in a sunscreen composition having an alkyl benzoate. One or more emollients may also be added at a given amount to replace a portion of an alkyl benzoate. For example, caprylic/capric triglycerides may be present in an amount to offset C₁₂-C₁₅ alkyl benzoate that is reduced from 15 wt. %., such as down to 5.0 wt. % and up to 10 wt. %.

In one example, caprylic/capric triglycerides may be present in an amount between 0.1 wt. % to 14.9 wt. %, preferably between 5.0 wt. % to 10.0 wt. %, including every number and/or fraction therebetween such as 1.0 wt. %, 2.0 wt. %, 3.0 wt. %, 4.0 wt. %, 5.0 wt. %, 6.0 wt. %, 7.0 wt. %, 8.0 wt. %, 9.0 wt. %, 10.0 wt. %, 11.0 wt. %, 12.0 wt. %, 13.0 wt. %, 14.0 wt. %, 14.9 wt. %, in combination with C₁₂-C₁₅ alkyl benzoate in an amount of greater than or equal to 0.1 wt. % and less than 15.0 wt. %, preferably between 5.0 wt. % and 10 wt. %, based on the total weight of the sunscreen composition. In a further example, dimethicone may be present, for example in an amount between about 0.1 wt. % to about 3.5 wt. %, which may enhance anti-foaming properties of the sunscreen composition.

Humectant(s)

A sunscreen composition may include a humectant to absorb water from the air or an underlying keratinous substrate, and/or draw water molecules toward the surface of the keratinous substrate. Preferably, the humectant includes a lipid such as, without limitation, glycerin. Additionally or alternatively, the humectant may be synthesized from two or more different monomers to form copolymers and/or from individual polymer chains that are connected by bridging molecules or crosslinking agents to form crosslinked copolymers known as crosspolymers. Thus, for example, the humectant may include octyldodecanol citrate crosspolymer itself and/or with other constituent monomers. In one example, the humectant may include stearyl/octyldodecyl citrate crosspolymer, which is the copolymer of stearyl alcohol, octyldodecanol, and citric acid crosslinked with 1,3-propanediol.

A humectant may be present in an amount of about 1.0 wt. % to about 20.0 wt. %, based on the total weight of the sunscreen composition. A humectant may, for example, be present in an amount of 0.9 wt. %, 2.0 wt. %, 3.0 wt. %, 7.0 wt. %, 10.0 wt. %, 15.0 wt. %, 22.0 wt. %, including every number and/or fraction therebetween, based on the total weight of the sunscreen composition. For example, a sunscreen composition may include glycerin and/or stearyl/octyldodecyl citrate crosspolymer present in an amount between about 1.5 wt. % to about 7.5 wt. %, based on the total weight of the sunscreen composition.

Emulsifier(s)

A sunscreen composition may include an emulsifier to stabilize an emulsion. The emulsifier may include a nonionic emulsifier, a cationic emulsifier, an anionic emulsifier, an amphoteric emulsifier, or combinations thereof. An emulsifier may include a water-in-oil (W/O) emulsifier having a hydrophilic-lipophilic balance (HLB) of less than 6, preferably from 4 to 6. Examples of useful W/O emulsifiers in embodiments include, without limitation. certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), glycerin and/or fatty acid esters (e.g., glyceryl stearate, etc.), or combinations thereof.

Meanwhile, an oil-in-water (O/W) emulsifier may have a HLB of greater than 7, preferably 7 to 10. Examples of oil-in-water (0/W) emulsifiers in embodiments include, without limitation. certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB greater than 8, etc. Useful natural emulsifiers may include, without limitation, cetearyl alcohol (and) cetearyl glucoside, glyceryl stearate (and) cetearyl alcohol (and) sodium stearoyl lactylate, cetearyl wheat straw glycosides (and) cetearyl alcohol, sorbitan olivate, cetearyl olivate (and) sorbitan olivate, or combinations thereof. Additionally or alternatively, an emulsifier may include a dispersion including a mineral, a triglyceride, a solvent, or combinations thereof. For example, the emulsifier may include a dispersion of caprylic/capric triglyceride, stearalkonium bentonite, and propylene carbonate.

An emulsifier may be present in an amount of about 1.0 wt. % to about 15.0 wt. %, based on the total weight of the sunscreen composition. An emulsifier may, for example, be present in an amount of 0.9 wt. %, 1.0 wt. %, 2.5 wt. %, 4.0 wt. %, 5.0 wt. %, 7.5 wt. %, 11.0 wt. %, 16.5 wt. %, including every number and/or fraction therebetween, based on the total weight of the sunscreen composition. For example, a sunscreen composition may include cetyl polyethylene glycol (PEG)/polypropylene glycol (PPG)-10/1 dimethicone, lauryl PEG-8 dimethicone, and/or a dispersion of caprylic/capric triglyceride, stearalkonium bentonite, and propylene carbonate present in an amount between about 2.0 wt. % to about 10.0 wt. %, based on the total weight of the sunscreen composition.

Film Former(s)

A sunscreen composition may include a film former to provide a substantially homogenous and/or structured hydro-lipidic film on skin owing to its viscosity-building properties. Generally, a film former may be synthesized from two or more different monomers to form copolymers and/or from individual polymer chains that are connected by bridging molecules or crosslinking agents to form crosslinked copolymers known as crosspolymers. Moreover, a film former may be present in a non-aqueous solvent such as, for example, an alcohol, an ether, a fatty acid, a silicone fluid, an ester, a hydrocarbon, a fluorinated hydrocarbon, or combinations thereof. In one example, a film former is dissolved in isododecane, isohexadacane, diisopropyl adipate, isoparrafins (e.g., C₁₃-C₁₆ isoparrafins), alcohol (e.g., C₂-C₁₀ alcohols, etc.), organosilicon compounds (e.g., disiloxane, dimethicone, cyclopentasiloxane, etc.), or combinations thereof.

A film former may be selected from monomers, copolymers, cross polymers, and/or terpolymers of an organo-silicone hybrid, biopolymer, (meth)acrylate, a bietic acid derivative, olefin, silicone, resin, vinyl acetate (VA), vinyl pyrrolidone (VP), maleate, alkyl ester, long chain or short chain carboxylic acid, or combinations thereof. A film former may include, without limitation, crotonic acid/vinyl C₈₋C₁₂ isoalkyl esters/VA/bis-vinyldimethicone crosspolymer, acrylate/dimethicone copolymer, VA/butyl maleate/isobornyl acrylate copolymer, methyl dihydroabietate, trimethylsiloxysilicate, acrylate/C₁₂₋C₂₂ (meth)acrylate copolymer, C₂₄₋C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, shellac, stearyl/octyldodecyl citrate crosspolymer or combinations thereof.

A film former may be present in an amount of about 0.1 wt. % to about 5.0 wt. %, based on the total weight of the sunscreen composition. A film former may, for example, be present in an amount of 0.09 wt. %, 0.9 wt. %, 1.0 wt. %, 2.0 wt. %, 3.0 wt. %, 4.0 wt. %, 5.0 wt. %, 5.5 wt. %, including every number and/or fraction therebetween, based on the total weight of the sunscreen composition. For example, a sunscreen composition may include VP/eicosene copolymer or stearyl/octyldodecyl citrate crosspolymer present in an amount less than about 4.0 wt. %, based on the total weight of the sunscreen composition.

Other Ingredient(s)

A sunscreen composition may optionally include or intentionally exclude other ingredients such as, without limitation, added water, cosmetically acceptable carriers, oils, sterols, amino acids, moisturizers, powders, colorants, pigments, dyes, pH adjusters, fragrances, cosmetic active ingredients, vitamins, preservatives, preservative boosters, foaming agents, chelating agents, cleansers, dispersing agents, thickeners, stabilizers (e.g., light, emulsion, etc.), essential fatty acids, sphingolipids, self-tanning compounds, fillers, antioxidants, surfactants, additional film formers, chelating agents, gelling agents, minerals, viscosity and/or rheology modifiers, keratolytics, retinoids, hormonal compounds, alpha-keto acids, anti-mycobacterial agents, anti-fungal agents, anti-microbials, anti-virals, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, pharmaceutical agents, spherical powders, extracts (fruit, flower, plant), absorbents, salicylic acid, alpha and beta hydroxy acids, retinol and its derivatives, or combinations thereof.

One or more other ingredients may be present in an amount of about 0.01 wt. % to about 99.0 wt. %, based on the total weight of the sunscreen composition. One or more other ingredients may, for example, be present in an amount of 0.009 wt. %, 0.1 wt. %, 0.5 wt. %, 1.0 wt. %, 5.0 wt. %, 10.0 wt. %, 20.0 wt. %, 30.0 wt. %, 35.0 wt. %, 40.0 wt. %, 50.0 wt. %, 75.0 wt. %, 99.5 wt. %, including every number and/or fraction therebetween, based on the total weight of the sunscreen composition. For example, a sunscreen composition may include a preservative system, a thickener, a solvent, an extract, a colorant, a stabilizer, a dispersing agent, and/or a fragrance present in an amount between about 0.01 to about 45.0 wt. %, based on the total weight of the sunscreen composition. A sunscreen composition may also intentionally exclude and/or may not require one or more other ingredients such as, without limitation, an emulsion stabilizer (e.g., starches, pectins, gums, etc.), and so on.

Sunscreen Format(s)

A sunscreen composition may include one or more formulation formats such as, without limitation, an emulsion (O/W, W/O, etc.), a gel, an oil, an anhydrous composition, a base, or combinations thereof. Additionally or alternatively, a sunscreen composition may include one or more application formats such as, without limitation, a lotion, a cream, a spray, a stick, a mouse, a foam, a powder, an ointment, or combinations thereof. A sunscreen composition may, for example, be formulated as a concentrate with a propellant to provide a continuous aerosol spray commercial product. The propellant generally exists as equilibrium of vapor and liquid and can be either dissolved in or miscible with the composition. Examples of suitable propellants include isobutene, butane, propane, dimethyl 10 ether, methyl ether, or combinations thereof. The aerosol propellant may be present in an amount of about 10 wt. % to about 60 wt. %, based on a total weight of the sunscreen composition (e.g., final composition). A bag-on-valve system may also be used to deliver the sunscreen composition using compressed gas such as air, nitrogen, or carbon dioxide. In another example, the sunscreen composition may be formulated as an aqueous lotion. Preferably, a sunscreen composition incudes a sun milk application format.

### Example(s)

All equipment was cleaned and sanitized before batching. Ingredients were mixed in as phases according to traditional known techniques. Example Table 1 shows example sunscreen compositions having relatively high concentrations of mineral sunscreen actives together with emollient(s), humectant(s), emulsifier(s), film former(s), and/or other ingredient(s) that exhibited markedly improved drag, rub-in, and spreadability attributes.

TABLE 1

| Component (INCI) | Function | Approx. wt. % |
|---|---|---|
| Example Main Ingredients | | |
| Zinc Oxide and/or Titanium Oxide | Sunscreen Active | 3.0-20.0 |
| C₁₂-C₁₅ Alkyl Benzoate, Oil, Dimethicone, Glyceryl Stearate, | Emollient | 0.1-30.0 |
| Caprylic/Capric Triglycerides, and/or Butyloctyl Salicylate | | |
| Glycerin and/or Stearyl/Octyldodecyl Citrate Crosspolymer | Humectant | 1.5-7.5 |
| Cetyl PEG/PPG-10/1 dimethicone, Lauryl PEG-8 Dimethicone, | Emulsifier | 2.0-10.0 |
| and/or Caprylic/Capric Triglyceride (and) Stearalkonium | | |
| Bentonite (and) and Propylene Carbonate | | |
| VP/Eicosene Copolymer or Stearyl/Octyldodecyl Citrate | Film Former | 0.1-4.0 |
| Crosspolymer | | |
| Example Other Ingredients | | |
| Phenoxyethanol (and) Ethylhexylglycerine, Sodium Chloride, | Preservative | 0.1-5.0 |
| Flower Extract, Fruit Extract, Bismuth Oxychloride (and) Mica | System, Thickener, | |
| (and) Iron Oxide, Mica (and) Iron Oxides (and) Titanium | Extract, Colorant, | |
| Dioxide, Ethylhexyl Methoxycrylene, and/or Polyhydrostearic | Light Stabilizer, | |
| Acid | Dispersing Agent | |
| Water, Alcohol (denatured) | Solvent | Q.S. to 100 |

Various example sunscreen compositions from Table 1 were compared to a commercially available mineral sunscreen lotion (Comparative Example 1) to evaluate sensory attributes for applicability and/or performance. Testing was based on the ASTM E 1490-03 Standard Practice for Descriptive Skinfeel Analysis of Creams and Lotions.

As shown in Example Table 2, Comparative Example 1 lacked C₁₂-C₁₅ alkyl benzoate and included about 11 wt. % zinc oxide and titanium dioxide. Examples 1-4, however, included C₁₂-C₁₅ alkyl benzoate present in an amount of 15.0 wt. % or more, based on the total weight of the sunscreen composition. Examples 1-4 further included one or more other emollients selected from oil (plant-derived), dimethicone, glyceryl stearate, and/or butyloctyl salicylate, present in an amount between about 0.1 wt. % to about 6.0 wt. %, based on the total weight of the sunscreen composition. Examples 1-4 may, for example, include dimethicone in an amount between about 0.1 wt. % to about 3.5 wt. %. Examples 1-4 may further optionally include caprylic/capric triglycerides, for example to account for decreases in C₁₂-C₁₅ alkyl benzoate, for example below 15.0 wt. % to about 5.0 wt. %.

Examples 1-4 further included a humectant selected from glycerin and stearyl/octyldodecyl citrate crosspolymer, present in an amount between about 1.5 wt. % to about 7.5 wt. %, based on the total weight of the sunscreen composition. Examples 1-4 further included an emulsifier selected from cetyl PEG/PPG-10/1 dimethicone, lauryl PEG-8 dimethicone, and/or caprylic/capric triglyceride (and) stearalkonium bentonite (and) and propylene carbonate, present in an amount between about 2.0 wt. % to about 10.0 wt. %, based on the total weight of the sunscreen composition. Examples 1-4 further included VP/eicosene copolymer or stearyl/octyldodecyl citrate crosspolymer present in an amount less than about 4.0 wt. %, based on the total weight of the sunscreen composition.

Examples 1-4 included water in an amount of 30.0 wt. % or more, based on the total weight of the sunscreen composition. Examples 1-4 further included one or more other ingredients selected from phenoxyethanol (and) ethylhexylglycerine, sodium chloride, alcohol, extract (fruit, flower, etc.), bismuth oxychloride (and) mica (and) iron oxide, mica (and) iron oxides (and) titanium dioxide, ethylhexyl methoxycrylene, and/or polyhydrostearic acid, present in an amount between about 0.01 wt. % to about 5.0 wt. %, based on the total weight of the sunscreen composition. Examples 1-4 optionally did not include an emulsion stabilizer such as starches, pectins, gums, and so on.

TABLE 2

| | | Relative | Attributes (immediately after rub in) | | | |
|---|---|---|---|---|---|---|
| | Approx. wt. % | C₁₂-C₁₅ | | | | Sticky/ |
| Examples | of Active | Alkyl Benzoate | Rub-In | Spreadability | Thickness | Tackiness |
| Comparative | 11% ZnO/TiO₂ | None | 3 | 2 | 4 | 4 |
| Example 1 | | | | | | |
| Example 1 | 11.5% ZnO/TiO₂ | Highest | 6 | 7 | 2 | 2 |
| Example 2 | 10% ZnO/TiO₂ | Next Highest | 5 | 6 | 2.5 | 2.5 |
| Example 3 | 20% ZnO | Lowest | 5.5 | 5 | 3 | 2 |
| Example 4 | 15% ZnO | Next Lowest | 5 | 5 | 3.5 | 2.5 |

As shown in Table 2, a rating difference of greater than one (1) point signifies a statistically significant change that is of practical sensory importance. Notably, there was a two-fold sensory improvement in rub-in for Example 1, having the highest amount of C₁₂-C₁₅ alkyl benzoate within the range in Table 1, compared to Comparative Example 1, which lacked C₁₂-C₁₅ alkyl benzoate, despite having more mineral sunscreen active. Surprisingly, rub-in decreased at most by one point, from a rating of 6 to a rating of 5, with less C₁₂-C₁₅ alkyl benzoate used in Examples 2-4 compared to Example 1. For example, the improvement in rub-in compared to Comparative Example 1 was maintained with about 28% less C₁₂-C₁₅ alkyl benzoate in Example 3 compared to Example 1 despite nearly doubling the total amount of mineral sunscreen active. A three-fold improvement in spreadability compared to Comparative Example 1 was also observed for Example 1 compared to Comparative Example 1 and continued to be markedly improved for Examples 2-4. Additionally, thickness showed a two-fold improvement for Example 1 compared to Comparative Example 1 and continued to be markedly improved for Example 2 with no worse results for Examples 3-4, while sticky/tackiness also showed a two-fold improvement for Example 1 compared to Comparative Example 1 and continued to be markedly improved for Examples 2-4 despite using less C₁₂-C₁₅ alkyl benzoate.

Accordingly, relatively high concentrations of mineral sunscreen actives (e.g., >10.0 wt. %) may not induce drag in a sunscreen composition, cause a sunscreen composition to be relatively harder to smooth into skin, and/or cause a sunscreen composition to be relatively harder to spread onto skin. Moreover, sensory attributes indicative of application and/or performance such rub-in, spreadability, thickness, and/or sticky/tackiness may be enhanced despite relatively high concentrations of mineral sunscreen actives, relatively high concentration of water, relatively low concentrations of humectant, or combinations thereof.

Additional Notes and Examples

Additional Example 1. includes a sunscreen active including a metal oxide, wherein the metal oxide is present in an amount between about 3.0 wt. % to about 20.0 wt. %, based on 25 the total weight of the composition, and an emollient including a benzoate ester, wherein the benzoate ester is present in an amount between about 5.0 wt. % to about 30.0 wt. %, based on the total weight of the composition.

Additional Example 2 includes the sunscreen composition of Additional Example 1, wherein the metal oxide is comprises zinc oxide, titanium dioxide, or combinations thereof.

Additional Example 3 includes the sunscreen composition of any of Examples 1-2, wherein the metal oxide is selected from the group consisting of zinc oxide.

Additional Example 4 includes the sunscreen composition of any of Examples 1-3, wherein the benzoate ester includes C₁₂-C₁₅ alkyl benzoate.

Additional Example 5 includes the sunscreen composition of any of Examples 1-4, wherein the benzoate ester is present in an amount of 15 wt. % or more, based on the total weight of the composition.

Additional Example 6 includes the sunscreen composition of any of Examples 1-5, wherein the emollient further includes one more emollients other than the benzoate ester selected from a diol, an oil, a fat, a lipid, a triglyceride, a siloxane, salicylic acid and/or derivatives thereof, or combinations thereof.

Additional Example 7 includes the sunscreen composition of any of Examples 1-6, wherein the one more emollients other than the benzoate ester includes plant-based oil, dimethicone, glyceryl stearate, butyloctyl salicylate, or combinations thereof.

Additional Example 8 includes the sunscreen composition of any of Examples 1-7, wherein the one or more emollients other than the benzoate ester are present in an amount between about 0.1 wt. % to about 6.0 wt. %, based on the total weight of the composition.

Additional Example 9 includes the sunscreen composition of any of Examples 1-8, wherein the benzoate ester includes C₁₂-C₁₅ alkyl benzoate present in an amount between about 5.0 wt. % to about 10.0 wt. %, based on the total weight of the composition, and wherein the emollient further includes caprylic/capric triglycerides in an amount between about 5.0 wt. % to about 10.0 wt. %, based on the total weight of the composition.

Additional Example 10 includes the sunscreen composition of any of Examples 1-9, further including a humectant.

Additional Example 11 includes the sunscreen composition of any of Examples 1-10, wherein the humectant is selected from glycerin, octyldodecyl citrate crosspolymer, or combinations thereof.

Additional Example 12 includes the sunscreen composition of any of Examples 1-11, wherein the humectant is present in an amount between about 1.0 wt. % to about 20.0 wt. %, based on the total weight of the composition.

Additional Example 13 includes the sunscreen composition of any of Examples 1-12, wherein the humectant is present in an amount between about 1.5 wt. % to about 7.5 wt. %, based on the total weight of the composition.

Additional Example 14 includes the sunscreen composition of any of Examples 1-13, further including an emulsifier.

Additional Example 15 includes the sunscreen composition of any of Examples 1-14, The composition of any of the preceding claims, wherein the emulsifier is selected from a nonionic emulsifier, a cationic emulsifier, an anionic emulsifier, an amphoteric emulsifier, or combinations thereof.

Additional Example 16 includes the sunscreen composition of any of Examples 145, wherein the emulsifier is selected from cetyl PEG/PPG-10/1 dimethicone, lauryl PEG-8 dimethicone, caprylic/capric triglyceride (and) stearalkonium bentonite (and) and propylene carbonate, or combinations thereof.

Additional Example 17 includes the sunscreen composition of any of Examples 1-16, wherein the emulsifier is present in an amount between about 2.0 wt. % to about 10.0 wt. %, based on the total weight of the composition.

Additional Example 18 includes the sunscreen composition of any of Examples 1-17, further including a film former.

Additional Example 19 includes the sunscreen composition of any of Examples 148, wherein the film former is selected from monomers, copolymers, cross polymers, and/or terpolymers of an organo-silicone hybrid, biopolymer, (meth)acrylate, a bietic acid derivative, olefin, silicone, resin, vinyl acetate (VA), vinyl pyrrolidone (VP), maleate, alkyl ester, long chain or short chain carboxylic acid, or combinations thereof.

Additional Example 20 includes the sunscreen composition of any of Examples 1-19, wherein the film former is selected from VP/eicosene copolymer, octyldodecyl citrate crosspolymer, or combinations thereof.

Additional Example 21 includes the sunscreen composition of any of Examples 1-20, wherein the film former is present in an amount between about 0.1 wt. % to about 5.0 wt. %, based on the total weight of the composition.

Additional Example 22 includes the sunscreen composition of any of Examples 1-21, further including added water, cosmetically acceptable carriers, oils, sterols, amino acids, moisturizers, powders, colorants, pigments, dyes, pH adjusters, fragrances, cosmetic active ingredients, vitamins, preservatives, preservative boosters, foaming agents, chelating agents, cleansers, dispersing agents, thickeners, stabilizers, essential fatty acids, sphingolipids, self-tanning compounds, fillers, antioxidants, surfactants, additional film formers, chelating agents, gelling agents, minerals, viscosity and/or rheology modifiers, keratolytics, retinoids, hormonal compounds, alpha-keto acids, anti-mycobacterial agents, anti-fungal agents, anti-microbials, anti-virals, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, pharmaceutical agents, spherical powders, extracts (fruit, flower, plant), absorbents, salicylic acid, alpha and beta hydroxy acids, retinol and its derivatives, or combinations thereof.

Additional Example 23 includes the sunscreen composition of any of Examples 1-22, further including a preservative system, a thickener, a solvent, an extract, a colorant, a stabilizer, a dispersing agent, or combinations thereof.

Additional Example 24 includes the sunscreen composition of any of Examples 1-23, wherein water is present in an amount of 30 wt. % or more, based on the total weight of the composition.

Additional Example 25 includes a method to protect a keratinous surface from UV radiation comprising contacting the keratinous surface with the sunscreen composition of any of Examples 1-24.

Additional Example 26 includes a method to form the sunscreen composition of any of Examples 1-24, comprising mixing the sunscreen active including the metal oxide and the emollient including the benzoate ester, and optionally further comprising mixing one more emollients other than the benzoate ester, a humectant, an emulsifier, a film former, one or more other ingredients, or combinations thereof.

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

1-20. (canceled)

21. A composition comprising:
a sunscreen active selected from the group consisting of a metal oxide, wherein the metal oxide comprises zinc oxide, titanium dioxide, or combinations thereof, and wherein the metal oxide is present in an amount between about 3.0 wt. % to about 20.0 wt. %, based on the total weight of the composition;
an emollient including:
C₁₂-C₁₅ alkyl benzoate present in an amount between about 5.0 wt. % to less than 30.0 wt. %, based on the total weight of the composition; and
dimethicone present in an amount between about 3.0 wt. % to about 3.5 wt. %, based on the total weight of the composition;
an emulsifier present in an amount between about 2.0 wt. % to about 10 wt. %, based on the total weight of the composition; and
water present in an amount of 30.0 wt. % or more, based on the total weight of the composition.

22. The composition of claim 21, wherein the metal oxide is selected from the group consisting of zinc oxide.

23. The composition of claim 21, wherein the C₁₂-C₁₅ alkyl benzoate is present in an amount of 15.0 wt. % or more, based on the total weight of the composition.

24. The composition of claim 21, wherein the emollient further includes a diol, an oil, a fat, a lipid, a triglyceride, salicylic acid and/or derivatives thereof, or combinations thereof.

25. The composition of claim 24, wherein the emollient includes plant-based oil, glyceryl stearate, butyloctyl salicylate, or combinations thereof.

26. The composition of claim 21, wherein the C₁₂-C₁₅ alkyl benzoate is present in an amount between about 5.0 wt. % to about 10.0 wt. %, based on the total weight of the composition, and wherein the emollient further includes caprylic/capric triglycerides present in an amount between about 5.0 wt. % to about 14.9 wt. %, based on the total weight of the composition.

27. The composition of claim 21, further including a humectant.

28. The composition of claim 27, wherein the humectant is selected from glycerin.

29. The composition of claim 27, wherein the humectant is present in an amount between about 1.5 wt. % to about 7.5 wt. %, based on the total weight of the composition.

30. The composition of claim 21, wherein the emulsifier is selected from the group consisting of a water-in-oil emulsifier.

31. The composition of claim 30, wherein the water-in-oil emulsifier is selected from cetyl polyethylene glycol (PEG)/ polypropylene glycol (PPG)-10/1 dimethicone, lauryl PEG-8 dimethicone, or combinations thereof.

32. The composition of claim 21, further including a film former.

33. The composition of claim 32, wherein the film former is selected from monomers, copolymers, cross polymers, and/or terpolymers of an organo-silicone hybrid, biopolymer, (meth)acrylate, abietic acid derivative, olefin, silicone, resin, vinyl acetate (VA), vinyl pyrrolidone (VP), maleate, alkyl ester, long chain or short chain carboxylic acid, or combinations thereof.

34. The composition of claim 32, wherein the film former includes VP/eicosene copolymer, octyldodecyl citrate crosspolymer, or combinations thereof.

35. The composition of claim 32, wherein the film former is present in an amount between about 0.1 wt. % to about 5.0 wt. %, based on the total weight of the composition.

36. The composition of claim 21, further including cosmetically acceptable carriers, oils, sterols, amino acids, moisturizers, powders, colorants, pigments, dyes, pH adjusters, fragrances, cosmetic active ingredients, vitamins, preservatives, preservative boosters, foaming agents, chelating agents, cleansers, dispersing agents, thickeners, stabilizers, essential fatty acids, sphingolipids, self-tanning compounds, fillers, antioxidants, surfactants, film formers, chelating agents, gelling agents, minerals, viscosity and/or rheology modifiers, keratolytics, retinoids, hormonal compounds, alpha-keto acids, anti-mycobacterial agents, anti-fungal agents, anti-microbials, anti-virals, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, staining agents, depigmenting agents, hypopigmenting agents, pharmaceutical agents, spherical powders, extracts, absorbents, salicylic acid, alpha and beta hydroxy acids, retinol and its derivatives, or combinations thereof.

37. The composition of claim 21, wherein the composition excludes an emulsion stabilizer selected from the group consisting of starches, pectins, and gums.

38. The composition of claim 21, wherein the composition is in the form of a non-foaming lotion.

39. A method to protect a keratinous surface from ultraviolet (UV) radiation comprising contacting the keratinous surface with the composition of claim 1.

40. The method of claim 39, wherein contacting the keratinous surface includes rubbing in the composition of claim 1.
